(19) United States
(12) Patent Application Publication
(10) Pub. No.: US 2004/0147876 A1
(43) Pub. Date: Jul. 29, 2004

(54) Disposable retractable-needle syringe

(76) Inventor: Tarcisio Maggioni, Milano (IT)

Correspondence Address: James V Costigan, Hedman & Costigan, Suite 2003, 1185 Avenue of the Americas, New York, NY, 10036-2601, US

(21) Appl. No.: 10/476,725

(22) PCT Filed: May 2, 2001
(86) PCT No.: PCT/IT01/00210

(51) Int. Cl. A61M 5/00

(52) U.S. Cl. 604/110

(57) ABSTRACT

A disposble rectractable-needle syringe comprises a cylindric body including a first end for engaging therein a piston element and a second end having an attachmen for a needle assembly. The piston element comprises latching means for latching the needle assembly and allowing the needle to be introduced into the cylindric body upon ending the injection operation, by operating the piston element. The latching means comprise one end of the gasket applied to the piston, designed for receiving hook means included in the needle assembly.

### BACKGROUND OF THE INVENTION

The present invention relates to a disposable retractable-needle syringe.

As is known, in using hypodermic needles, it is absolutely necessary to prevent a needle from transmitting infective diseases.

In fact, the syringe needle may contact the skin of the sanitary operator, and this also due to a repeated use of the syringe.

Several approaches have been submitted to reduce the problems related to the disposal of disposable syringes and from preventing said disposable syringes-from being reused.

Among the proposed approaches, retractable needle syringes have been designed, in which, upon use, the syringe needle is withdrawn into the syringe cylindric body, by resilient means.

Such a syringe, however, has the drawback that it cannot be used by conventional methods, since it compels the operator to overcome the counter-biassing force of the mentioned resilient means in performing the injection operation.

Another drawback is that said syringes have a comparatively high cost, due to their constructional complexity.

Yet other types of disposable syringes comprise collapsable coupling means connecting the syringe needle to the syringe cylindric body; on the other hand, it has been found that these latter syringes are scarcely reliable in operation.

### SUMMARY OF THE INVENTION

Accordingly, the aim of the present invention is to provide a disposable retractable-needle syringe, which is adapted to overcome the above mentioned drawbacks of prior retractable-needle syringes.

Within the scope of the above mentioned aim, main object of the present invention is to provide a disposable syringe including a retractable needle, specifically designed for preventing said syringe from being reused, and which is constructionally very simple and very inexpensive from a mere making standpoint.

A further object of the present invention is to provide a retractable needle disposable syringe, which does not include any resilient elements, and which can be used in a manner fully like to that of a conventional syringe.

Yet a further object of the present invention is to provide such a disposable retractable-needle syringe, the components of which can be assembled by automatic assembling apparatus, thereby minimizing any manual operations by human operators, and which, moreover, has very good hygienic characteristics.

Yet a further object of the present invention is to provide such a disposable retractable-needle syringe which can be made by conventional elements used for making conventional syringes, possibly with very simple modifications allowing to use conventional syringe molding apparatus and molds.

According to one aspect of the present invention, the above mentioned aim and objects, as well as yet other objects, which will become more apparent hereinafter, are achieved by a disposable retractable-needle syringe, comprising a syringe body including a first end for engaging therein a piston element and a second end including an attachment for a needle assembly, characterized in that said piston element comprises latching means for latching said needle assembly, designed for allowing the syringe needle to be introduced into said cylindric body upon ending an injection operation, by operating said piston element.

The latching means comprise one end of the gasket of the piston element, designed for receiving hook means provided in the needle assembly.

### BRIEF DESCRIPTION OF THE DRAWINGS

Further characteristics and advantages of the present invention will become more apparent hereinafter from the following detailed disclosure of a preferred, though not exclusive, embodiment of the invention, which is illustrated, by way of an indicative, but not limitative, example, in the accompanying drawings, where:

FIG. 1 is an exploded view of the disposable retractable-needle syringe according to the present invention;

FIG. 2 is an enlarged partial view of that same disposable retractable-needle syringe, being shown in a piston feeding operation thereof, said piston having a gasket to which is applied an insert element including teeth designed for latching the needle assembly;

FIG. 3 is a view analogous to FIG. 2, in which the syringe is shown at the end of stroke position of the syringe piston in which the needle has been latched;

FIG. 4 is a further schematic view of the disposable retractable-needle syringe according to the present invention, shown in a piston feeding operation, said piston being provided with a gasket in which are directly formed the needle latching teeth or annular elements;

FIG. 5 is a view similar to FIG. 4, in which the syringe is illustrated in an end of stroke position of the piston with the needle in a latched condition;

FIG. 6 is a further schematic view illustrating the syringe as the syringe piston is rearward driven with the needle latched thereto;

FIG. 7 is a further schematic view illustrating the syringe with the syringe needle fully withdrawn and the piston being broken;

FIG. 8 is a further schematic view of the disposable retractable-needle syringe, according to a further aspect of the present invention, being shown during a piston feeding operating step; and

FIG. 9 is a schematic view similar to FIG. 8, in which the syringe of FIG. 8 is shown in an end of stroke position of the piston with the needle being latched.

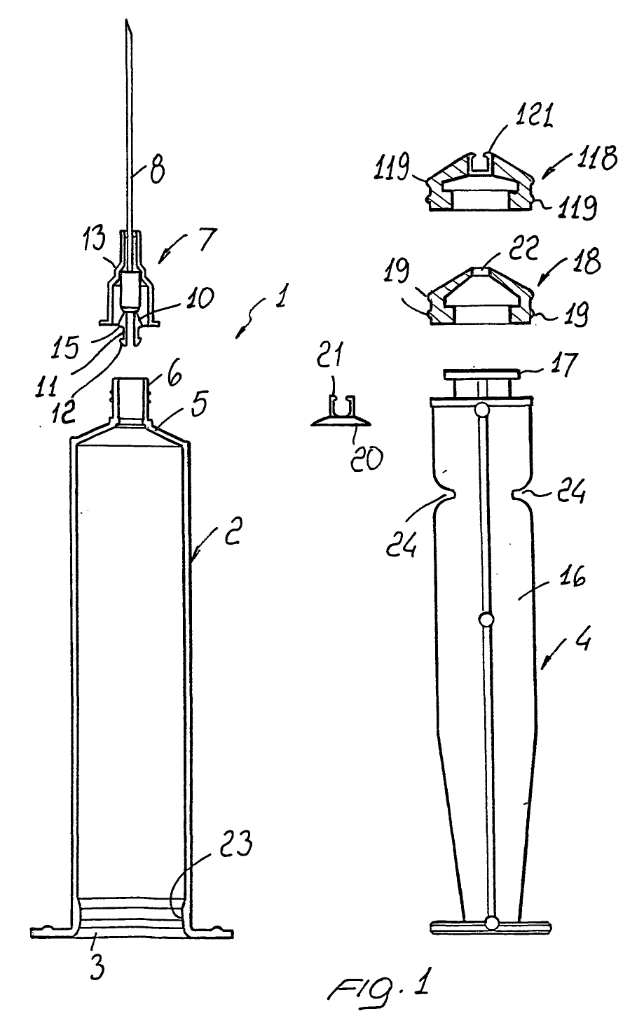

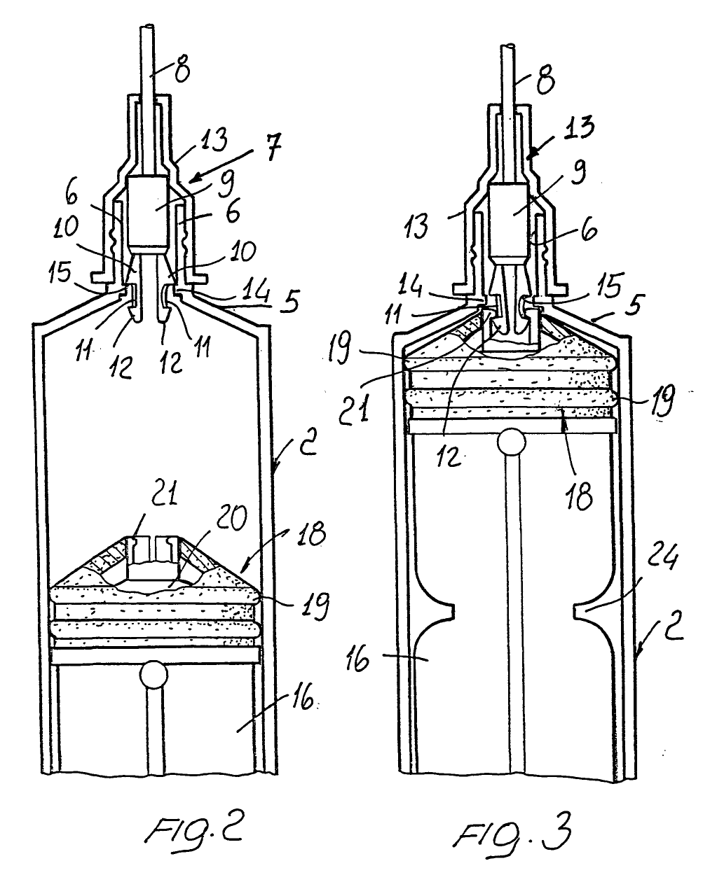

### DESCRIPTION OF THE PREFERRED EMBODIMENTS

With reference to the number reference of the accompanying drawings, the disposable retractable-needle syringe according to the present invention, generally indicated by the reference number 1, comprises a syringe cylindric body 2, having a first end 3, for engaging therein a piston element 4, and a second end 5 including an end-piece 6 operating as an attachment element for a needle assembly 7.

Advantageously, at said first syringe end 3 is provided a narrowed region 23, preventing the piston element 4 from being disengaged from the cylindric body.

The cylindric body 2 and piston element 4 can have any conventional configuration, without any specifically designed constructional modification, with respect to the same components of a conventional syringe.

The needle assembly 7 comprises a needle 8, including an end portion opposite to the tip coupled to a small cylinder 9.

The latter comprises resilient wings 10, each of which is provided with an outer cut-out 11, designed for defining a latching tooth element 12 and an abutment 15.

The needle assembly 7 comprises a cap 13, for holding therein said elements and engaging the end-piece 6 of the cylindric body 2 in order to lock the needle assembly 7 at a set use position thereof.

The inner portion of the end-piece 6 comprises an annular step portion 14, designed for engaging the abutment 15 of the wings 10 to counter-bias the pushing force generated during the injection operation and for holding the needle at the use position thereof, and for preventing said needle from being accidentally withdrawn.

The piston element 4 comprises a stem 16, including an attachment 17 for a sealing gasket 18, 118.

According to one aspect of the present invention, the sealing gasket 18 comprises a rubber element including annular ridges 19 designed for providing a sealing relationship with the inner wall of the cylindric body 2, in a per se known manner.

The sealing gasket 18 is hollow and is adapted to engage the attachment element 17 and restrain an insert 20, having an end portion 21 projecting from a hole 22 formed in the gasket 18.

Said end portion 21 is provided for receiving the teeth 12 of the wings 10 of said needle assembly 7.

According to a modified embodiment of the invention, the sealing gasket 118 comprises a rubber element including annular ridges 119 for sealing against the inner wall of the cylindric body 2, in a per se known manner.

The sealing gasket 118 is hollow, and is designed for engaging the attachment element 17 and being provided with an end portion 121 for engaging therein and latching the teeth 12 of the wings 10 of said needle assembly 7.

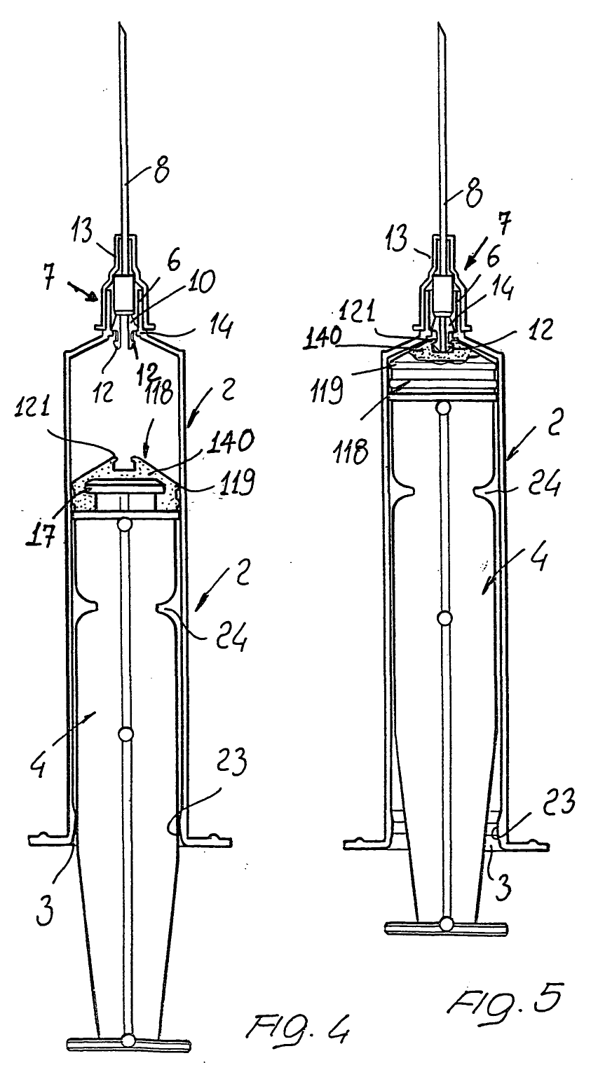

FIGS. 4, 5 and 6, 7 illustrate operating steps for using the syringe according to the present invention, which is suitable to perform an injection operation by applying, in a conventional manner, a pressure on the piston element 4, with a consequent delivery of the product or medicament to be injected.

Upon ending the delivery operation, the end portion 21, 121 of the gasket applied to the piston 4 will engage said resilient wings 10 of the needle assembly, by clamping them and thereby disengaging the abutment 15 of the wings from the annular step portion 14 of the cylindric body.

Thus, the needle assembly will be engaged by the gasket or inserts of the gasket applied to the piston.

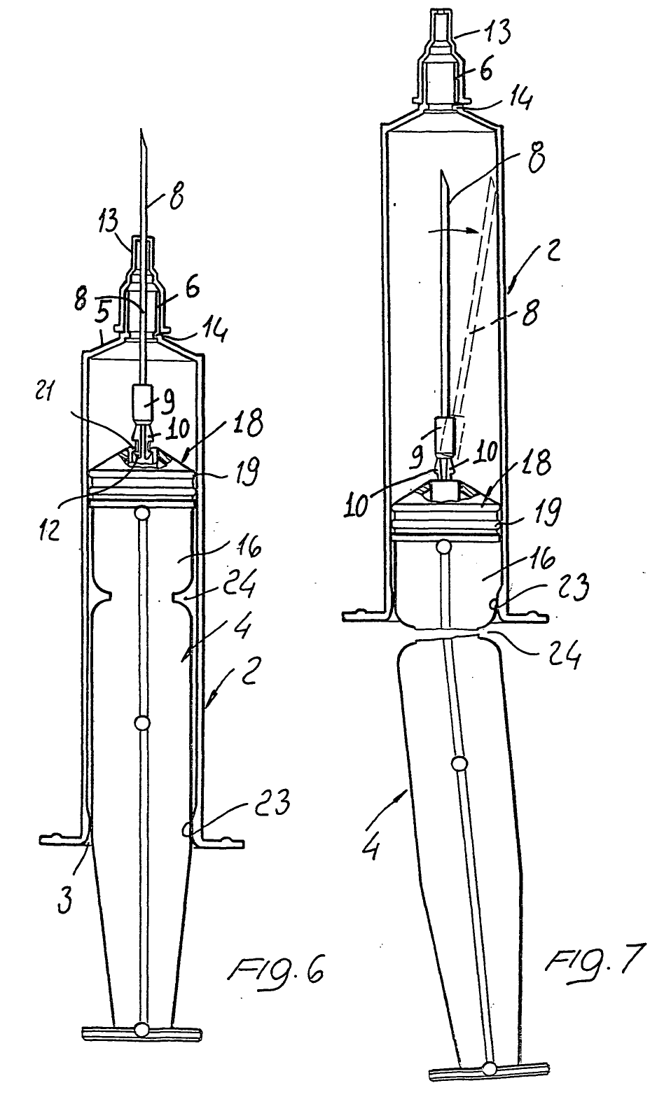

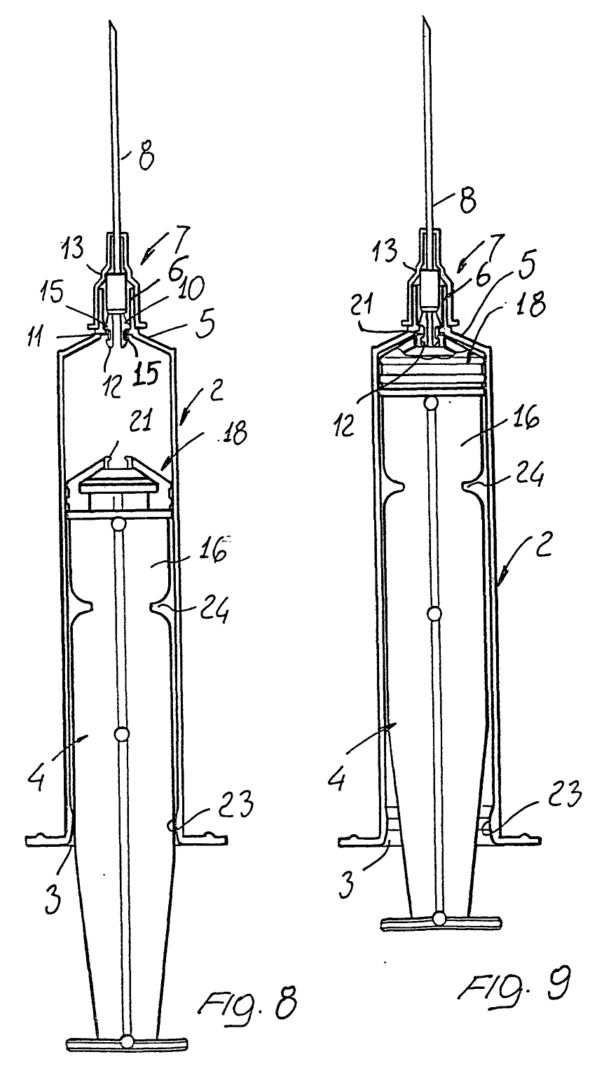

By withdrawing the piston element (FIG. 6), the needle is withdrawn inside the cylindric body and, as it has been fully withdrawn inside said cylindric body, it will assume an inclined position (FIG. 7), due to a difference of the two cut-outs 11 of the wings 10.

Thus, any further pressure applied to the piston 4 will not be able of further withdrawing the needle.

As the piston 4 is arranged outside the cylindric body 2, it would be moreover possible to break said piston, owing to the provision of suitable notches 24, thereby further preventing any possible reuse of the syringe.

The subject syringe is:characterized in that the needle 8 is rigid with the small cylinder 9 and resilient wings 10, which latter project outward, in an opposite direction.

Said resilient wings 10, in particular, are provided with opposite cut-outs 11, thereunder it is possible to see latching teeth 12 outward projecting and having a contour designed for spreading apart and engaging suitable teeth 21 inward projecting, rigid with an insert 20 coupled to a sealing gasket 18, 118, in turn provided with annular ridges 19, 119 for providing a sealing relationship against the inner wall of the cylindric body 2.

Alternatively, and as is shown in FIGS. 4 and 5, the sealing gasket 140 could be provided with an end portion 121 for directly engaging and latching the teeth 12 of the resilient wings 10.

The disposable syringe according to the present invention comprises a needle assembly 7 including a cap 13 for holding said small cylinder 9 having resilient wings 10, and designed for engaging the end-piece 6 of the cylindric body 2 for locking at a use position the needle assembly 7.

At this time, the syringe cannot be further used, and the syringe needle will be fully withdrawn inside the cylindric body.

Thus, the syringe can be safely handled and disposed of.

It has been found that the invention fully achieves the intended aim and objects.

Actually, a disposable retractable-needle syringe has been provided, which can be made by substantially using existing syringe making apparatus, already used for making conventional syringes.

A further advantage of the invention is that the subject syringe can be assembled by automatic assembling machine, thereby providing a perfectly hygienic product, in addition to a much higher production yield.

The disposable retractable-needle syringe according to the present invention is susceptible to several modifications and variations, all of which will come within the scope of the inventive idea.

All the constructional details can be replaced by other technically equivalent elements.

In practicing the invention, the used materials, and the contingent size and shapes, can be any, according to requirements and the status of the art.

1. A disposable retractable-needle syringe (2), comprising a syringe cylindric body (2) with an end-piece including a first end (3) for engaging therein a piston element (4) having a piston stem and a second end (5) provided with an attachment (6) for a needle assembly (7), said piston element (4) comprising latching means for latching said needle assembly (7) and allowing the needle (8) to be introduced into said syringe cylindric body (2) upon ending an injection operation, by operating said piston element (4), said latching means comprising an end portion of a hollow sealing gasket (18, 118, 140) applied to said piston element (4), designed for receiving hook means provided in said needle assembly (7), said needle assembly (7) comprising a needle (8) including an end opposite to a tip portion associated with a small cylinder (9) having two outward projecting resilient wings (10), each said resilient wing (10) being provided with an outer cut-out (11) designed for defining a latching tooth and an abutment, said piston stem having an attachment element (17) for engaging said sealing gasket (18, 118, 140), said sealing gasket comprising annular ridges for sealing against an inner wall of said cylindric body, said sealing gasket being designed for engaging with said attachment element and holding an insert element comprising an end portion projecting from a hole formed in said gasket for engaging said teeth of said two resilient wings (10) of said needle assembly, an inner portion of said end-piece comprising an annular step portion for engaging said abutment of said wings to counterbias a pushing force generated by an injection operation and for holding said needle in said use position thereof, to prevent said needle from being accidentally withdrawn, characterized in that at said first end of said syringe is provided a narrowed region (23) preventing said piston element (4) from being disengaged from said cylindrical body (2), that said sealing gasket is made of a rubber material, that said two wings (10) are provided each with a respective cut-out (11), said cut-outs being so constructed and arranged that, as they are fully engaged by said gasket and said piston element (4) is fully withdrawn inside said cylindrical body, also said needle (8) will be fully withdrawn inside said cylindrical body and will assume an inclined position, and that said piston (4) is provided with two peripheral breaking notches (24) to allow said piston to be broken as said piston projects for a set length from said cylindrical body (2) to further prevent said syringe from being used.

2. A disposable retractable-needle syringe, according to claim 1, characterized in that said needle assembly (7) comprises a cap (13) for holding therein said elements and engaging said end-piece of said cylindric body (2) for locking said needle assembly (7) at a use position thereof.

3. A disposable retractable-needle syringe, according to claims 1 and 2, characterized in that said needle (8) is rigid with said small cylinder (9) in turn made rigid with said two resilient wings (10).

4. A disposable retractable-needle syringe, according to one or more of the preceding claims, characterized in that under said cut-outs (11) of said wings (10) are formed said outward projecting latching teeth and having a contour designed for engaging with and spreading apart inward projecting annular elements, formed on or made rigid with said sealing gasket.
